(19) United States
(12) Patent Application Publication
(10) Pub. No.: US 2011/0270170 A1
(43) Pub. Date: Nov. 3, 2011

(54) Steerable Medical Device Having Means For Imparting Multiple Curves in the Device

(75) Inventors: Kenneth C. Gardeski, Plymouth, MN (US); Ronald Alan Drake, St. Louis Park, MN (US)

(73) Assignee: Medtronic, Inc., Minneapolis, MN (US)

(21) Appl. No.: 12/771,333

(22) Filed: Apr. 30, 2010

(51) Int. Cl. A61M 25/092; A61M 25/00

(52) U.S. Cl. 604/95.04; 604/524

(57) ABSTRACT

A steerable elongated medical device that is capable of forming as “s” shaped bend. The devices include an outer tube extending between an outer tube proximal segment and an outer tube distal segment, having an outer tube wall forming an outer tube lumen and first and second elongated outer tube slots through the outer tube wall to the outer tube lumen. The first elongated outer tube slot has a first portion and a second portion and formed between a first outer tube slot proximal end and a first outer tube slot distal end and extending axially along the outer tube distal segment through a first outer tube slot length to define a first cutaway portion of the outer tube. The second elongated outer tube slot has a first portion and a second portion and formed between a second outer tube slot proximal end and a second outer tube slot distal end and extending axially along the outer tube distal segment through a second outer tube slot length to define a second cutaway portion of the outer tube and offset about 180 degrees from the first elongated outer tube slot. Each of the first and second outer tube slots have reinforcing sleeves, each of the reinforcing sleeves having reinforcing sleeve slot portions and reinforcing sleeve overlap portions.

### FIELD

The present invention pertains to use of elongated medical instruments to access a site in the body to facilitate introduction of a further medical device, and particularly to elongated steerable medical devices for steering the distal end and imparting curves in distal segments of such medical instruments to facilitate implantation of a medical device.

### BACKGROUND

A wide variety of elongated medical instruments are currently available that are adapted to be permanently or temporarily implanted in the mammalian body, usually the body of a human patient, or used to access a site in the body to facilitate introduction of a further implantable medical device or delivery of a therapeutic or diagnostic agent. Such elongated medical instruments have an instrument body extending between instrument body proximal and distal ends, and a distal segment of the instrument body is advanced to a remote site in the body.

In many cases, the introduction of such elongated medical instruments to a remote site in the body is effected through a skin incision accessing an incision into a blood vessel, whereby the instrument body is advanced through a pathway until the distal segment or the instrument body distal end are located at the remote site. Such advancement is often through a tortuous pathway having twists and turns requiring the capability to impart a curve or deflect the instrument body distal end to facilitate advancement. Therefore, the introduction of such elongated medical instruments through vascular pathways or other tortuous pathways in the body is facilitated by a wide variety of techniques and mechanisms that have been developed to impart curves in the distal segment of the instrument body or to deflect or steer the instrument body distal end.

There is still a perceived need for a steerable medical device having a small diameter medical device body that is simple and inexpensive to manufacture, resists kinking, and that can be manipulated to control the deflection of and impart a bend in a distal segment of the medical device body that is more acute.

### SUMMARY

In one embodiment, the invention includes a steerable elongated medical device, that comprises an outer tube extending between an outer tube proximal segment and an outer tube distal segment, having an outer tube wall forming an outer tube lumen and first and second elongated outer tube slots through the outer tube wall to the outer tube lumen, the first elongated outer tube slot having a first portion and a second portion and formed between a first outer tube slot proximal end and a first outer tube slot distal end and extending axially along the outer tube distal segment through a first outer tube slot length to define a first cutaway portion of the outer tube, the second elongated outer tube slot having a first portion and a second portion and formed between a second outer tube slot proximal end and a second outer tube slot distal end and extending axially along the outer tube distal segment through a second outer tube slot length to define a second cutaway portion of the outer tube and offset about 180 degrees from the first elongated outer tube slot;

a first reinforcing sleeve positioned within the outer tube lumen and extending between a first reinforcing sleeve proximal end and a first reinforcing sleeve distal end, wherein the first reinforcing sleeve forms a reinforcing sleeve slot portion aligned with and extending along the first portion of the first outer tube slot and includes a reinforcing sleeve overlap portion extending over the second portion of the first outer tube slot; and

a second reinforcing sleeve positioned within the outer tube lumen and extending between a second reinforcing sleeve proximal end and a second reinforcing sleeve distal end, wherein the second reinforcing sleeve forms a reinforcing sleeve slot portion aligned with and extending along the first portion of the second outer tube slot and includes a reinforcing sleeve overlap portion extending over the second portion of the first outer tube slot.

In another embodiment, the invention includes steerable elongated medical device, comprising an outer tube extending between an outer tube proximal segment and an outer tube distal segment, having an outer tube wall forming an outer tube lumen and first and second elongated outer tube slots through the outer tube wall to the outer tube lumen, the first elongated outer tube slot having a first portion and a second portion and formed between a first outer tube slot proximal end and a first outer tube slot distal end and extending axially along the outer tube distal segment through a first outer tube slot length to define a first cutaway portion of the outer tube, the second elongated outer tube slot having a first portion and a second portion and formed between a second outer tube slot proximal end and a second outer tube slot distal end and extending axially along the outer tube distal segment through a second outer tube slot length to define a second cutaway portion of the outer tube and offset about 180 degrees from the first elongated outer tube slot;

a first reinforcing sleeve positioned within the outer tube lumen and extending between a first reinforcing sleeve proximal end and a first reinforcing sleeve distal end, wherein the first reinforcing sleeve forms a reinforcing sleeve slot portion aligned with and extending along the first portion of the first outer tube slot and includes a reinforcing sleeve overlap portion extending over the second portion of the first outer tube slot;

a second reinforcing sleeve positioned within the outer tube lumen and extending between a second reinforcing sleeve proximal end and a second reinforcing sleeve distal end, wherein the second reinforcing sleeve forms a reinforcing sleeve slot portion aligned with and extending along the first portion of the second outer tube slot and includes a reinforcing sleeve overlap portion extending over the second portion of the first outer tube slot;

a handle coupled to the outer tube proximal end; and a pull wire positioned within the outer tube lumen and extending between a pull wire proximal end coupled to the handle and a pull wire distal end coupled to a pull wire stop, the pull wire extending through the first and second reinforcing sleeve lumens and the outer tube lumen, wherein the pull wire proximal end is adapted to be manipulated to separate the pull wire proximal end from the outer tube proximal end to induce a bend in each of the first and second cutaway portions.

### BRIEF DESCRIPTION OF THE DRAWINGS

FIG. 1 is a simplified perspective view of a steerable elongated medical device in which an embodiment of the present invention can be incorporated;

FIG. 2 is a simplified top view of a distal segment of an elongated medical device body having first and second elongated outer tube slots reinforced with first and second inner reinforcement sleeves and a guidewire tip in accordance with a first embodiment of the present invention;

FIG. 3 is a simplified side view of the distal segment of the elongated medical device body of FIG. 2:

FIG. 4 is a cross-section view taken of an embodiment of elongated medical device handle shown in FIG. 1;

FIG. 5 is a simplified side view of a distal segment of a elongated medical device body having first and second elongated outer tube slots reinforced with an inner reinforcement sleeve in accordance with a second embodiment of the present invention;

FIG. 6 is a cross-sectional view of the distal segment depicted in FIG. 5.

FIG. 7 is a representation of a medical device of the invention within vessels of a heart; and

FIG. 8 is a representation of a distal portion of a medical device of the invention anchored within a vessel.

### DETAILED DESCRIPTION

In the following detailed description, references are made to illustrative embodiments for carrying out the invention. It is understood that other embodiments may be utilized without departing from the scope of the invention. Although the present invention is described as preferably being applied to a steerable medical device, the aspects of the present invention are not intended to be limited to use in steerable medical devices. Rather, it is understood that the present invention could be utilized in elongated medical devices other than steerable medical devices, such as sheaths or catheters, for example. Embodiments of the steerable medical devices of the present invention can be employed within the lumens of endocardial cardiac leads, particularly endocardial pacing and/or cardioversion/defibrillation leads, but it will be understood that the steerable medical devices of present invention can be utilized in any elongated medical instruments of the types described or mentioned herein and equivalents that may presently exist or come into existence in the future.

Moreover, the steerable medical devices of the present invention can advantageously be fabricated in a relatively large diameter for use in directing introducers or guide catheters through tortuous pathways, for example, for implanting cardiac leads that do not have lead lumens. Steerable medical device bodies for insertion into cardiac lead lumens typically have a medical device body outer diameter of about 0.016 inches. Larger diameter steerable medical devices having an outer diameter of 0.022 inches, for example, (incorporating the reinforcing sleeve construction of the present invention) can advantageously be employed in guide lumens of such bilumen catheters having a guide lumen diameter exceeding the typical cardiac lead body lumen diameter. It is not easier to create more robust steerable medical devices or medical devices that form smaller radii bends in the bendable distal sections of larger diameter (e.g., 0.022 inches) steerable medical device bodies than in smaller diameter (e.g., 0.016 inches) steerable medical devices. Examples of bilumen catheters that can be guided and/or anchored by utilizing the medical devices described in this application in a guide lumen of a bilumen catheter are describe in U.S. patent application publication No. 2004/0116878, incorporated by reference in this application for its description of bilumen guide catheters.

Moreover, the larger diameter steerable medical device has sufficient torqueability to increase torqueability of certain guide catheter shafts or bodies or to impart torquebility to a guide catheter shaft or body lacking any torqueability. In addition, it is inherently easier and less expensive to fabricate such larger diameter steerable medical device bodies insertable into a guide lumen of a catheter body than to fabricate smaller diameter steerable medical devices providing the same performance.

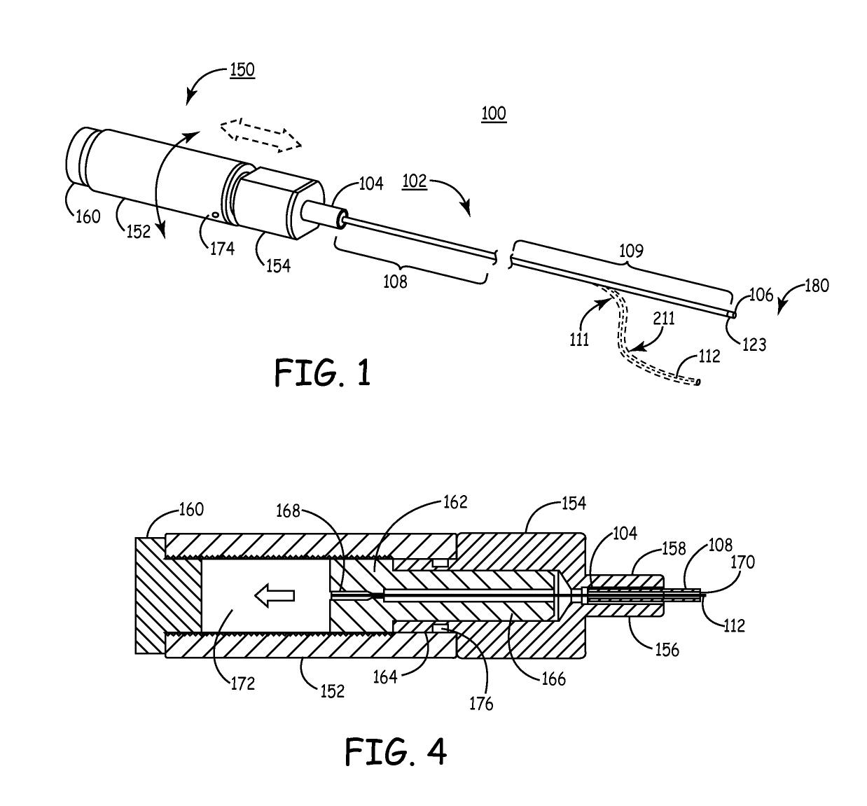

According to one embodiment, an elongated medical instrument, such as a steerable medical device 100, that includes a handle 150 and an elongated medical device body 102 in which embodiments and features of the present invention can advantageously be combined in various combinations is depicted in FIG. 1. The medical device body 102 extends a predetermined length between a medical device body proximal end 104 coupled to handle 150 and a medical device body distal end 106. The medical device body 102 further includes a medical device body proximal segment 108 and a medical device body distal segment 109. The medical device body 102 encloses an elongated pull wire 112 extending between a pull wire proximal end coupled to handle 150 and a pull wire distal end affixed at or near the medical device body distal end 106 or abutting a shoulder of the medical device body distal end 106. For example, the pull wire distal end is coupled to cylindrical distal pull wire stops 123 that abut a shoulder of the medical device body distal end 106 as described further below.

As shown in FIG. 1, the medical device handle 150 includes a proximal handle portion 152 and a distal handle portion 154 where proximal handle portion 152 rotates relative to distal handle portion 154 to induce first and second bends in the medical device body distal segment 109. For example, the pull wire proximal end is coupled to the handle insert 162 (shown in FIG. 4) and the medical device body proximal end 104 is coupled to the distal handle portion 154. The pull wire extends distally to a medical device wire distal end that is coupled with the medical device body distal end 106. The proximal handle portion 152 can be rotated and causes the pull wire attached to the handle insert 162 to be tensioned (but not twisted), and first and second bends, e.g., an “S” shaped bend depicted in broken lines depicted in FIG. 1, can be induced in the medical device body distal segment 109 in planes defined by first cutaway or slot portion 111 and second cutaway or slot portion 211. While the term “pull wire” is used herein, it can be seen that in practice, the medical device body proximal end is pushed away from the pull wire proximal end. The induced bend remains even if the grip on the proximal and distal handle portions 152 and 154 is released. First and second slot portions are spaced apart from each other and are offset as described in more detail below.

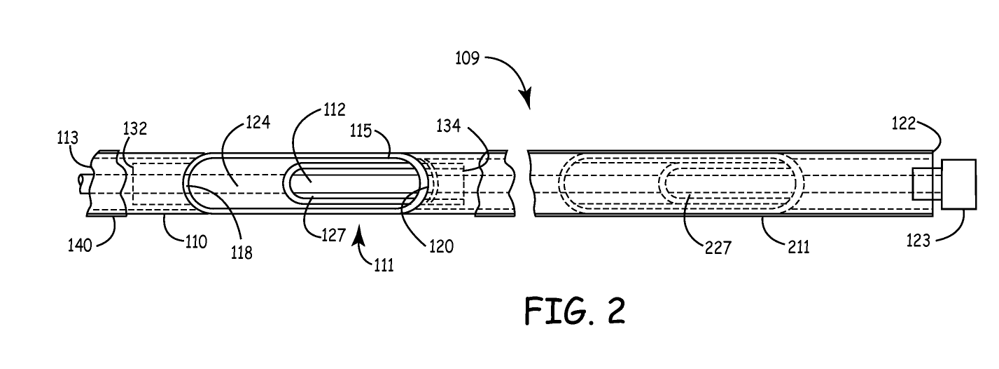

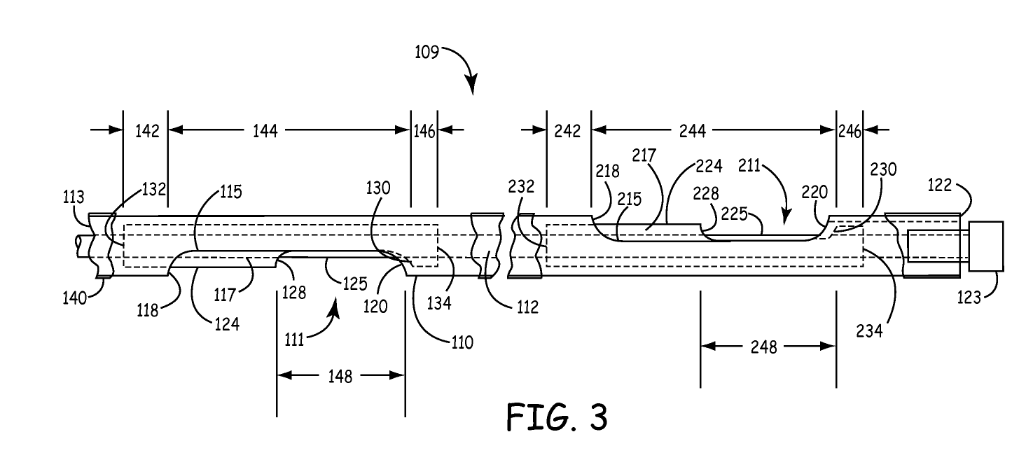

One embodiment of the distal segment 109 of device body 102 is depicted in FIGS. 2 and 3. In this embodiment, an outer tube 110 extends the full length of the medical device body 102 through the proximal and distal segments 108 and 109 to an outer tube distal end 122. The outer tube 110 is preferably formed of stainless steel hypodermic needle or “hypotube” or a shape memory alloy, e.g., NITi alloy or NITINOL brand alloy, having an outer diameter of about 0.022 inches and an outer tube wall thickness of about 0.005 inches, providing an outer tube lumen 113 having an outer tube lumen diameter of about 0.012 inches.

Offset elongated portions of the outer tube wall are cut away through an arc of about 180° along proximal and distal cutaway portions 111, 211 near the medical device body distal end. As shown in FIG. 3, the first elongated outer tube slot 115 is formed between first outer tube slot proximal end 118 and first outer tube slot distal end 120. The second elongated tube slot 215 is formed between second outer tube slot proximal end 218 and second outer tube slot distal end 220. In this embodiment shown in FIGS. 2 and 3, first and second elongated tube slots 115, 215 are offset by 180°.

First and second reinforcement tubes or sleeves 124, 224 are fitted into the outer tube lumen 113, each extending at least partly through the length of the first and second outer tube slots 115, 215. First and second reinforcement sleeves 124, 224 each have a reinforcement sleeve length extending between first and second reinforcement sleeve proximal ends 132, 232 and first and second reinforcement sleeve distal ends 134, 234, the lengths of the reinforcement sleeves 124, 224 exceeding the length of the outer tube slots 115, 215. First and second reinforcement sleeves 124, 224 form reinforcing sleeve lumens 127, 227 so that the reinforcing sleeves 124, 224 have an outer diameter to fit the dimensions of the outer tube lumen 113, for example, approximately 0.011 inches, and an inner diameter of for example, approximately 0.008 inches. The reinforcement sleeves are preferably formed of a shape memory alloy.

First and second reinforcement sleeves 124, 224 are fitted into the outer tube lumen 113 so that each reinforcement sleeve proximal segment 142, 242 extends proximally within outer tube lumen 113 from the proximal ends 118, 218 of the outer tube slots 115, 215 and each reinforcement sleeve distal segment 146, 246 extends distally within the outer tube lumen 113 from each distal end 120, 220 of the outer tube slots 115, 215. Each elongated reinforcement sleeve intermediate segment 144, 244 therefore extends the length of each outer tube slot 115, 215.

At least portions 148, 248 of each reinforcement sleeve intermediate segment 144, 244 are cut away through 180° to form first and second elongated reinforcement sleeve slots 125, 225 extending between reinforcement sleeve slot proximal ends 128, 228 and reinforcement sleeve slot distal ends 130, 230. Each outer tube slot 115, 215 and each reinforcement sleeve slot 125, 225 are aligned, and in this embodiment, each reinforcement sleeve proximal segment 142, 242 is bonded or adhered to the wall of the outer tube lumen 113 to maintain the alignment. The proximal segments may be bonded or adhered using an epoxy adhesive, for example those sold under the tradename “HYSOL”.

Bonding of the reinforcement sleeve proximal segments 142, 242 to the wall of the outer tube lumen 113 allows the reinforcement sleeve intermediate and distal segments 144, 244 and 146, 246 free to slide along a wall of the outer tube lumen 113 when a bend is induced along either or both cutaway portions 111, 211, such as by applying tension to the pull wire, for example. Overlap portions 117, 217 of each reinforcement sleeve proximal to the reinforcement sleeve slot proximal ends 128, 228 and that extends within the outer tube slots 115, 215 between each reinforcement sleeve slot proximal end 128, 228 and each outer tube slot proximal end 118, 228, provides strain relief bridging the outer tube slot proximal ends 118, 218 where buckling would otherwise likely take place.

In one embodiment, the length of the reinforcement sleeve proximal segments 142, 242 is approximately 0.100 to 0.250 inches, the length of the reinforcement sleeve intermediate segments 144, 244 is approximately 0.750 inches, and the length of the reinforcement sleeve distal segments 146, 246 is approximately 0.050 inches. The length of the reinforcement sleeve slots 125, 225 is approximately 0.300 inches. The reinforcement sleeve distal ends 134, 234 is, at a minimum, approximately 0.100 inches from the outer tube distal ends 122, 222. The length dimensions and ratio of the length of the reinforcement sleeve slots 125, 225 to the length of the reinforcement sleeve intermediate segment 144, 244 and the outer tube slot 115, 215 are be dependent upon the outer diameters, wall thicknesses, and materials of the outer tube and the reinforcement sleeve.

The ratio of the lengths of the solid wall reinforcement sleeve segments 142 and 146, 242 and 246 and the ratio of the cut away reinforcement sleeve segments 148, 248 with respect to the length of the first and second outer tube slot 115, 215 provide the optimal bending characteristics and resistance to kinking along the length of each first and second outer tube slot 115 and 215. Medical device bodies having varying flexibility and possible shapes formed in the distal segments thereof can be achieved by controlling the dimensions of each first and second outer tube slot 115 and 215 and each reinforcement sleeve slot 125 and 225. Generally, longer and deeper dimensions of the slots 125, 225 and 115, 215 increase flexibility but reduce rigidity in the cutaway portions 111 and 211. Thus, the dimensions of the slots 115, 215 and 125, 225 may be adjusted to provide the desired flexibility and rigidity of each cutaway portion 111 and 211 for the intended use.

In the illustrated embodiment, the slots 115, 215 and 125, 225 are shown to extend around the periphery of the outer tube 110 and the reinforcement sleeves 124, 224 in an about 180° arc leaving intact arcuate tube walls of the outer tube 110 and each reinforcement sleeve 124 and 224. The arc of the arcuate tube walls of the outer tube 110 and each reinforcement sleeve 124, 224 can differ from 180° and can differ from one another to achieve the desired flexibility and resulting bend shape.

The pull wire 112 extends through first and second reinforcement sleeve lumens 127 and 227 and the outer tube lumen 113 and terminates at pull wire stop 123 having a “stopper-like” shape. The distal end of the pull wire 112 is crimped or adhered into a lumen of a proximal extension of the pull wire stop 123 that fits into the outer tube lumen 113. The pull wire stop 123 has a distal end having an outer diameter approximately equal to the diameter of the outer tube 110, so that the pull wire stop 123 bears against the outer tube distal end 122 when stressed. In this way, the pull wire distal end is “coupled” to the outer tube distal end 122 at the medical device body distal end 106 as indicated in FIG. 1. The pull wire stop 123 can be adhesively bonded into the outer tube lumen 113 using an epoxy adhesive to inhibit ingress of bodily fluids.

Optionally, the pull wire 112 can be formed having a taper along its length, whereby the pull wire diameter extending through the first and second outer tube slots 115 and 215 and first and second reinforcement sleeve slots 125 and 225 is less than the pull wire diameter proximal to the aligned outer tube slot 115 and reinforcement sleeve slot 125. For example, the pull wire 112 can have a proximal wire diameter of 0.010 inches that is tapered to a distal wire diameter of 0.006 inches.

The shortened length of each cutaway portion 111, 211 achieved by use of the reinforcement sleeves 124, 224 also shortens the exposed length of the pull wire 112 so that it cannot extend as far away from the outer tube 110 when the outer tube 110 bows outward upon tensioning of the pull wire 112.

Optionally, a polymeric outer sleeve 140 is fitted over the outer tube 110 to extend over the first and second outer tube slots 115, 215 and first and second reinforcement sleeve slots 125, 225 to retain the pull wire 112 generally within the reinforcement sleeve lumens 127, 227 when the pull wire 112 is retracted to form a bend or bends in the nested outer tube and reinforcement sleeve walls. The polymeric outer sleeve 140 can be formed of polyimide or PEEK, for example, that is fitted or adhered over the outer tube 110.

In these ways, each reinforcement sleeve 124, 224 reinforces the wall of the outer tube 110 through at least a portion of the length of each outer tube slot 115, 215 to prevent buckling when a bend is induced in the outer tube 115 and in either or both reinforcement sleeves 124, 224. In this way, when the elongated medical instrument includes a pull wire, a bend can be safely induced in each of the remaining arcuate sections of the outer tube wall when the pull wire 112 extending through the outer tube lumen 113 is retracted with respect to the outer tube 110 (or the outer tube 110 is pushed distally with respect to the pull wire 112) by manipulation of the handle 150.

Referring again to FIG. 1, it is also possible to employ the principles of the present invention in a distal segment 109 of a medical device body 102 that is formed differently than the proximal segment 108 (or an intermediate segment) of the medical device body 102. The outer tube 110 can be replaced by a proximal outer tube segment and a distal outer tube segment formed of the same or differing materials, and having the same or differing wall outer tube diameters, wall thicknesses, and bending characteristics. The proximal and distal outer tube segments are joined together end-to-end, and the outer tube slot is formed in the tube wall of the distal tube segment.

For example, the proximal segment 108 of medical device body 102 can be formed from a proximal outer tube of stainless steel, for example, 304W stainless steel alloy, that is joined at a junction with a distal outer tube 110 that is formed of a shape memory alloy, e.g., NITINOL. In another embodiment, the proximal segment 108 can be formed of a wire braid reinforced polymer tube.

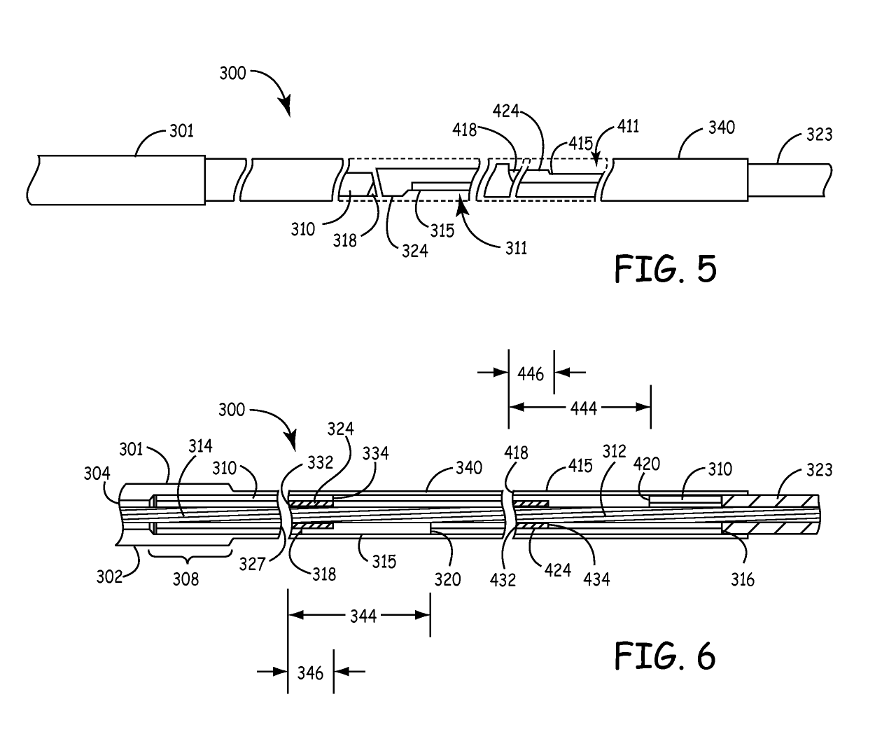

In the embodiment of medical device 300 depicted in FIGS. 5 and 6, the junction 308 is effected by an elongated distal counterbore 302 of the tube wall of the proximal outer tube 301 formed of stainless steel into which a proximal segment of the distal outer tube 310 is fitted and adhered employing epoxy cement or the like. A proximal outer tube lumen 304 is thereby axially aligned with a distal outer tube lumen 314. The proximal outer tube 301 preferably has an outer diameter of nominally 0.022 inches and an outer tube wall thickness of nominally 0.005 inches, providing an outer tube lumen 204 having a nominal outer tube lumen diameter of 0.012 inches. The counterbore diameter is preferably nominally 0.017 inches to receive a distal outer tube 310 having an outer diameter of nominally 0.016 inches and a distal outer tube lumen nominal diameter of 0.011 inches.

An elongated portion of the distal outer tube wall is cut away through 180° near the device body distal end between first distal outer tube slot proximal end 318 and first distal outer tube slot distal end 320 to form first elongated outer tube slot 315. Thus, an intact proximal tubular portion of the distal outer tube 310 extends proximally from the first distal outer tube slot proximal end 318, and an intact distal tubular portion of the distal outer tube 310 extends distally from first distal outer tube slot distal end 320.

An elongated portion of the distal outer tube wall is cut away through 180° near the device body distal end between second distal outer tube slot proximal end 418 and second distal outer tube slot distal end 420 to form second elongated outer tube slot 415. Thus, an intact proximal tubular portion of the distal outer tube 310 extends proximally from the second distal outer tube slot proximal end 418, and an intact distal tubular portion of the distal outer tube 310 extends distally from distal second outer tube slot distal end 420.

A first tubular distal reinforcement tube or sleeve 324 is fitted into the distal outer tube lumen 314 extending at least partly through the length of the first distal outer tube slot 315. The first reinforcement sleeve 324 is preferably formed of a shape memory alloy and has an outer sleeve outer diameter of 0.016 inches, for example, and a first reinforcement sleeve lumen 327 inner sleeve lumen diameter of 0.105 inches, for example.

A second tubular distal reinforcement tube or sleeve 424 is fitted into the distal outer tube lumen 314 extending at least partly through the length of the second distal outer tube slot 415. The second reinforcement sleeve 424 is preferably formed of a shape memory alloy and has an outer sleeve outer diameter of 0.016 inches, for example, and a first reinforcement sleeve lumen 327 inner sleeve lumen diameter of 0.105 inches, for example.

The reinforcement sleeves 324, 424 have a reinforcement sleeve length extending between first and second reinforcement sleeve proximal ends 332, 432 and first and second reinforcement sleeve distal ends 334, 434. In this embodiment, the reinforcement sleeves 324, 424 are tubular and do not have an elongated slot cut away leaving an arcuate section of the tubular wall. And, the reinforcement sleeves 324, 424 do not exceed the length of the first and second outer tube slots 315, 415. For example, the distal outer tube slots 315, 415 can have a nominal length of 1.500 inches, and the first and second reinforcement sleeve distal ends 334, 434 are disposed nominally at 0.375 to 0.750 inches from the distal outer tube slot proximal ends 318, 418 and distal outer tube slot distal ends 320, 420.

The pull wire 312 extends through the reinforcement sleeve lumen 327, the distal outer sleeve lumen 314 and the proximal outer sleeve lumen 304 to a cylindrical distal pull wire stop 323. The distal end of the pull wire 312 is crimped into a lumen of the distal pull wire stop 323. The cylindrical distal pull wire stop 323 has an outer diameter that exceeds the distal outer tube lumen 314 and bears against the distal outer tube distal end 316 when stressed.

Optionally, the pull wire 312 can be formed having a taper along its length, whereby the pull wire diameter extending through the distal outer tube slots 315, 415 and reinforcement sleeve lumen 327 is less than the pull wire diameter within the proximal outer tube lumen 304. For example, the pull wire 312 can have a proximal wire diameter of 0.010 inches that is tapered to a distal wire diameter of 0.006 inches.

Optionally, a polymeric outer sleeve 340 is fitted over the distal outer tube 310 to extend over the first and second distal outer tube slots 315, 415 and the first and second reinforcement sleeves 324, 424 to retain the pull wire 312 generally within the distal outer tube lumen 314 when the pull wire 312 is retracted to form a bend in the distal outer tube 310 and the first and second reinforcement sleeves 324, 424 in the first and second cutaway portions 311, 411. The polymeric outer sleeve 340 can comprise polyimide or PEEK that is fitted or adhered over the outer tube 310.

In these ways, the first and second reinforcement sleeves 324, 424 reinforces the wall of the distal outer tube 310 across the first and second outer tube slot proximal ends 318, 418 and through at least a portion of the full length of each of the distal outer tube slots 315, 415 to prevent buckling when a bend is induced in the distal outer tube 310 and each of the first and second reinforcement sleeves 324, 424 in the respective cutaway portions 311, 411. A bend can be safely induced in the arcuate section of the distal outer tube wall when the pull wire 312 extending through the aligned lumens is retracted with respect to the proximal and distal outer tubes 301 and 310 (or the outer tubes 301 and 310 are pushed distally with respect to the pull wire 312) by manipulation of handle 150.

The characteristics of the induced bends can be altered by the selection of the lengths 344, 444 of the first and second cutaway portions 311, 411, and the ratio of the each segment length 346, 446 to each full length 344, 444 of the respective first and second cutaway portions 311, 411. It should be noted that the reinforcement sleeves 324, 424 can also be shaped in the same manner as slotted reinforcement sleeves 124, 224 described above with respect to FIGS. 2 and 3. Additionally, reinforcement sleeve 124, 224 and 324, 424 may also be comprised of a wire coil, for example, such as the coil described in U.S. Pat. No. 6,146,338, incorporated by reference in this application for the description of a coil.

Referring again to Figure. 1, the medical device handle 150 can take any form that enables the application of tension between and to separate the proximal end of the pull wire 112 and the medical device body proximal end 104 apart to steer the medical device body distal end 106 and induce a bend along the cutaway portion 111.

One embodiment of a medical device handle 150 is shown in FIG. 4. The distal handle portion 154 has a tubular distal projection 156 into which the medical device body 104 is inserted and attached by adhesive. A distal handle portion lumen 158 extends through the distal handle portion 154. The proximal handle portion 152 is tubular and threaded internally within proximal handle portion lumen 172 at its proximal end to receive a handle end cap 160 and threaded more distally to receive the threads on the distal projection 168 of a handle insert 162 to enable the handle insert 162 to be moved distally to release tension or proximally to increase tension on the pull wire 112. The proximal end opening of the proximal handle portion 152 receives a tubular proximal projection 164 of the distal handle portion 154. The handle insert 162 comprises a handle insert proximal projection 166 extending into the distal handle portion lumen 158 and a threaded handle insert distal projection 168 that is attached to the internal threads of the proximal handle portion 152. The pull wire 112 is inserted through a handle insert lumen 170 that is axially aligned with the distal handle portion lumen 158 extending through the tubular distal projection 156. The proximal end of the pull wire 112 is attached to the handle insert 162.

One side of the handle insert distal projection 166 is flattened and bears against a guide pin (not shown) extending across the proximal handle lumen 172. A groove 176 is formed around the tubular proximal projection 164 to track a further guide pin (not shown). The user grips the sides of the distal handle portion 154 and rotates the proximal handle portion 152 to rotate the internal screw threads around lumen 172 with respect to the mating screw threads around handle insert distal projection 168 to move handle insert 162 proximally. The proximal movement tensions the pull wire 112 and induces the bend or bends as described above. Advantageously, the handle 150 is light in weight, and the tension and induced bend remains in place even if the handle 150 is released so that the user can manipulate the guide catheter handle without having to focus on manually maintaining the bend.

The above-described steerable medical devices advantageously facilitate the direct implantation of elongated cardiac leads having medical device lumens or the introduction and steering of guide catheters to a site of implantation of such cardiac leads or other medical devices or therapeutic or diagnostic substances.

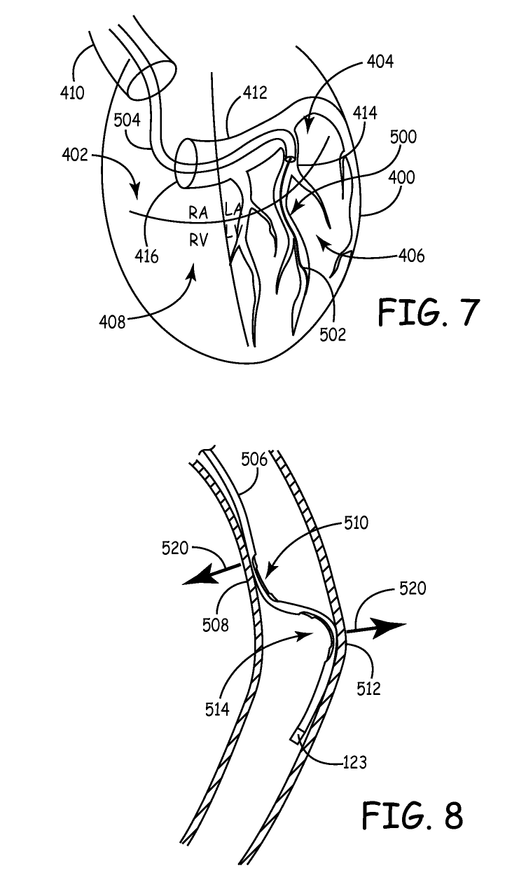

A depiction of the use of the steerable medical devices described in this application is shown in FIGS. 7 and 8. FIG. 7 depicts a partial view of a heart 400 including right and left atria 402 and 404, left and right ventricles 406 and 408, superior vena cava 410, and coronary sinus 412. The elongated body 502 of a steerable medical device 500 has been guided through a delivery catheter 504 to the posterior lateral vein 414. The delivery catheter 504 is guided through the superior vena cava 410 through the coronary sinus ostium 416, through the coronary sinus 412 and partially within the posterior lateral vein 414.

As shown in FIG. 8, the distal segment 506 of the elongated body 502 has a first bend 508 at the first slot portion 510 and a second bend 512 at the second slot portion 514. The outer tube walls that are opposite each of the first and second slot portions are pressed into the walls of the vessel via the opposing force as depicted by arrows 520.

While specific examples have been described in the specification and illustrated in the drawings, it will be understood by those of ordinary skill in the art that various changes may be made and equivalents may be substituted for elements thereof without departing from the scope of the present disclosure. Furthermore, the mixing and matching of features, elements and/or functions between various examples is expressly contemplated herein so that one of ordinary skill in the art would appreciate from this disclosure that features, elements and/or functions of one example may be incorporated into another example as appropriate, unless described otherwise, above. Moreover, many modifications may be made to adapt a particular situation or material to the teachings of the present disclosure without departing from the essential scope thereof. Therefore, it is intended that the present disclosure not be limited to the particular examples illustrated by the drawings and described in the specification but that the scope of the present disclosure will include any embodiments falling within the foregoing description.

1. A steerable elongated medical device, comprising:
an outer tube extending between an outer tube proximal segment and an outer tube distal segment, having an outer tube wall forming an outer tube lumen and first and second elongated outer tube slots through the outer tube wall to the outer tube lumen,
the first elongated outer tube slot having a first portion and a second portion and formed between a first outer tube slot proximal end and a first outer tube slot distal end and extending axially along the outer tube distal segment through a first outer tube slot length to define a first cutaway portion of the outer tube,
the second elongated outer tube slot having a first portion and a second portion and formed between a second outer tube slot proximal end and a second outer tube slot distal end and extending axially along the outer tube distal segment through a second outer tube slot length to define a second cutaway portion of the outer tube and offset about 180 degrees from the first elongated outer tube slot;
a first reinforcing sleeve positioned within the outer tube lumen and extending between a first reinforcing sleeve proximal end and a first reinforcing sleeve distal end, wherein the first reinforcing sleeve forms a reinforcing sleeve slot portion aligned with and extending along the first portion of the first elongated outer tube slot and includes a reinforcing sleeve overlap portion extending over the second portion of the first outer tube slot; and
a second reinforcing sleeve positioned within the outer tube lumen and extending between a second reinforcing sleeve proximal end and a second reinforcing sleeve distal end, wherein the second reinforcing sleeve forms a reinforcing sleeve slot portion aligned with and extending along the first portion of the second elongated outer tube slot and includes a reinforcing sleeve overlap portion extending over the second portion of the first outer tube slot.

2. The steerable medical device of claim 1 further comprising a polymeric outer sleeve fitted over the outer tube and extending over the first and second outer tube slots and the first and second reinforcement sleeve slots.

3. The steerable medical device of claim 1 wherein the first and second reinforcing sleeve overlap portions have a length that is a fraction of the first and second elongated outer tube slot lengths.

4. The steerable medical device of claim 1 wherein the first and second reinforcing sleeve overlap portions have a length that is approximately fifty percent of the first and second elongated outer tube slot lengths.

5. The steerable medical device of claim 1 wherein the second elongated tube slot has a length greater than the first elongated tube slot length.

6. A steerable elongated medical device, comprising:
an outer tube extending between an outer tube proximal segment and an outer tube distal segment, having an outer tube wall forming an outer tube lumen and first and second elongated outer tube slots through the outer tube wall to the outer tube lumen,
the first elongated outer tube slot having a first portion and a second portion and formed between a first outer tube slot proximal end and a first outer tube slot distal end and extending axially along the outer tube distal segment through a first outer tube slot length to define a first cutaway portion of the outer tube,
the second elongated outer tube slot having a first portion and a second portion and formed between a second outer tube slot proximal end and a second outer tube slot distal end and extending axially along the outer tube distal segment through a second outer tube slot length to define a second cutaway portion of the outer tube and offset about 180 degrees from the first elongated outer tube slot;
a first reinforcing sleeve positioned within the outer tube lumen and extending between a first reinforcing sleeve proximal end and a first reinforcing sleeve distal end, wherein the first reinforcing sleeve forms a reinforcing sleeve slot portion aligned with and extending along the first portion of the first outer tube slot and includes a reinforcing sleeve overlap portion extending over the second portion of the first outer tube slot;
a second reinforcing sleeve positioned within the outer tube lumen and extending between a second reinforcing sleeve proximal end and a second reinforcing sleeve distal end, wherein the second reinforcing sleeve forms a reinforcing sleeve slot portion aligned with and extending along the first portion of the second outer tube slot and includes a reinforcing sleeve overlap portion extending over the second portion of the first outer tube slot;
a handle coupled to the outer tube proximal end; and
a pull wire positioned within the outer tube lumen and extending between a pull wire proximal end coupled to the handle and a pull wire distal end coupled to a pull wire stop, the pull wire extending through the first and second reinforcing sleeve lumens and the outer tube lumen, wherein the pull wire proximal end is adapted to be manipulated to separate the pull wire proximal end from the outer tube proximal end to induce a bend in each of the first and second cutaway portions.

7. (canceled)

8. (canceled)

9. The device of claim 6 further comprising a polymeric outer sleeve fitted over the outer tube and extending over the first and second outer tube slots and the first and second reinforcement sleeve slots.

10. The device of claim 6 wherein the pull wire stop is bonded to the outer tube lumen.

11. The device of claim 9 wherein the polymeric outer sleeve is bonded to the outer tube.

12. A method of anchoring a steerable elongated medical device comprising:
guiding a distal portion of the steerable elongated medical device according to claim 6 to a site within a vasculature; and
inducing a bend in each of the first and second cutaway portions of the medical device, an outer tube wall opposite each of the cutaway portions being pressed against a vessel wall by opposing forces.
